(19) United States
(12) Patent Application Publication
(10) Pub. No.: US 2005/0170499 A1
(43) Pub. Date: Aug. 4, 2005

(54) Culture/exposure devices, kit for assembling a device of this type and method for cultivating and exposing prokaryotes

(76) Inventors: Ulrich Mohr, Hannover (DE); Franz Durst, Langensendelbach (DE)

Correspondence Address: Hooker & Habib, Suite 304, 100 Chestnut Street, Harrisburg, PA, 17101, US

(21) Appl. No.: 10/507,470

(22) PCT Filed: Mar. 14, 2003
(86) PCT No.: PCT/EP03/02711

(30) Foreign Application Priority Data
Mar. 14, 2002 (DE) 102113246

(51) Int. Cl. C12M 1/00; C12M 1/34

(52) U.S. Cl. 435/289.1; 435/288.3

(57) ABSTRACT

The invention relates to a culture/exposure device for accommodating cultures (72), comprising a system for subjecting the accommodated culture (72) to the action of a gaseous medium. The inventive device also comprises a mechanical flow guide (40) provided with an entry (42) for introducing the gaseous medium into the flow guide (40) and with an exit opening (44) that discharges above the surface of the culture (72). The flow guide (40) forcibly guides the gaseous medium. The invention is characterized in that the inner surface of the exit opening (44) of the flow guide (40) opens in a trumpet-like manner in the direction of flow. In addition, an intake tube (74, 74′) having an intake opening (76) for drawing in the gaseous medium is connected to the flow guide (40) and is characterized in that the inner surface of its intake opening (76) opens in a trumpet-like manner counter to the direction of flow. The invention also relates to a kit consisting of two different bases (2, 108) and two different tops (4; 96) that can be combined in any manner to form four different culture/exposure devices.

### BACKGROUND OF THE INVENTION

The invention concerns culture/exposure apparatuses for the receiving of cultures, a kit for the assembly of such culture/exposure apparatuses as well as a procedure for the culture of prokaryotes.

### DESCRIPTION OF THE PRIOR ART

As is disclosed in the state of the technology, culture/exposure apparatuses have been made known, wherein a cell culture contained within a culture/exposure apparatus is subjected to a gaseous medium. As an example thereof, the generically applicable publication DE 100 140 57 (assigned to assignee of the present invention) can be cited as well as EP 1 174 496.

The culture/exposure apparatus (disclosed in DE 100 140 57) for the reception of cell cultures, employs a culture container, which has a side wall structure conically tapering downward to its bottom with a increasing effective diameter. The said apparatus further is provided with means for subjecting the cell cultures to a gaseous environment, wherein the cell cultures can be treated under predetermined harmful or therapeutic conditions. For this purpose, gases, aerosols, and/or media bearing particulate can directly contact the cell cultures. Among the said gaseous media may be numbered, for example, tobacco smoke for its impingement upon lung cells. The exposure-apparatus encompasses for this purpose, a through-passage, cylindrically shaped, flow directional device. This device is so positioned in the said culture/exposure apparatus above the cell culture that a directed flow of the gaseous medium can be induced therethrough, flowing over the surface of the cell culture and through an annular opening between the said flow device and the wall of the culture container. Such a flow pattern, for example, is produced by a vacuum pump, which is placed in the path of the flow, downstream from the said annular opening. The flow duct, at the entering zone, is connected with a suction fitting, through which the ambient air, test gases or the like are subjected to vacuum and thus can be contactingly conducted over the cell cultures.

The cited EP 1 174 496 represents in the formation of the exposure apparatus, essentially, the stated publication of the present applicant. Moreover, this publication concerns itself in regard to more exactly determining the dosages of the entrained aerosol particles which deposit themselves on the cell culture. In this regard, the streamlining of the gas flow within the cylindrical flow duct is determined by visibly-marked aerosol particles noted in a pulsed laser beam. Subsequently, from the determined hyperbolically curved streamlines, those streamlines are selected, which travel at a defined distance above the cell culture surface and from which the entrained aerosols can still diffuse upon the cell culture surface. These streamlines are traced back to the entry of the flow duct and thereby an effective cross-section within this streamlining in reference to the entire cross-section of the opening of the flow duct is determined. By means of this effective cross-section, all through-flow aerosols can subject the surface of the cell culture to exposure.

Practical experience has demonstrated, that prior exposure apparatuses operate unsatisfactorily, because no continuous exchange of the gaseous medium above the cell culture surface exists and no uniformly contacting exposure of the cell cultures can be guaranteed. Obviously, above the cell culture surface, resident “dead zones” have formed. Further in the “exhausted gaseous medium” these dead zones have accumulated, wherein no fresh, gaseous medium can be directed to the cell cultures.

Also, problems have arisen in respect to the suction fittings, since, for example, the intake of ambient air by means of cylindrically shaped suction fittings is subjected to severe swings away from the predetermined suction intake capacity. On this account, no volume-specific suction is permitted and conversely, undesired suction variations from the vacuum lines occur.

Thus the invention has the purpose of optimizing the flow characteristics of the gas flow directed over the surface of the culture. Likewise, the invention also has the purpose of improving the suction based removal of a gaseous medium, for instance, from an outer chamber. Finally, another purpose of the invention is to make available extended research possibilities by the cultivation and exposure of prokaryotes.

### BRIEF SUMMARY OF THE INVENTION

The invention is a culture/exposure apparatus that optimizes the flow characteristics of the gas flow directed over the surface of the culture and improves the suction based removal of a gaseous medium.

A culture/exposure apparatus for the receiving of cultures with an arrangement for the treatment of the received culture with a gaseous medium in accordance with a first aspect of the present invention includes a mechanical flow duct with an entry for introducing a gaseous medium into the flow duct and an outflow opening placed above the surface of the culture whereby the flow duct directs the gaseous medium. The inner surface of the flow duct opens itself in a trumpet-like configuration.

Accordingly, a culture/exposure apparatus for the acceptance of cultures is created, which has a device for subjecting the resident culture with a gaseous medium. This said device possesses a mechanical generation of flow to compel the said gaseous medium to pass through an entry for the introduction of the said gaseous medium into the flow system. The device has further, an outlet opening placed above the surface of the culture. This outlet includes an inner, trumpet-like shaping which widens in the direction of the flow. The advantage of this opening which expands itself in a trumpetlike manner can be found in that, the flow release turbulence, which is especially difficult to control, is avoided at the edges of this type of opening. Normally, at the circumferential rim of a cylindrical discharge duct, turbulent eddies lead to the said dead zones above the culture. With the described special trumpetlike shaped exit opening, assurance is provided, that the flow of the gaseous medium is guided smoothly and free from turbulence over the surface of the culture. Further, the flow, including any therein entrained particles, is distributed to the greatest possible extent, uniformly over the culture surface.

It should be noted, that the concept “trumpet shaped” is not limited to circular configurations, but may encompass, for example, lengthened openings, (rectangular), square, polygonal, rotation-symmetrical or other opening cross-sections. The decisive factor is, principally, that the opening cross section maintains an increasing cross-section to the exit area. Conversely a linear outlet in the form of a conical frustum does not bring about the desired result, nor does any “tulip shaped” outlet.

Note should be taken that, the general concept of “culture” or “cell culture” encompasses, not only cell culture, but also the well known eukarotic cultures, the prokaryotic cultures and bacterial cultures and the like.

A culture/exposure apparatus for the reception of cultures with an apparatus for the exposing of the accepted culture with a gaseous medium in accordance with a second aspect of the present invention includes a suction fitting having a suction opening for the intake of the gaseous medium and an outlet that is connected with a flow guide for conducting the gaseous medium to a plane above the culture. The inner surface of the suction opening opens itself in a trumpetlike flare counter to the flow direction.

Accordingly, the culture/exposure apparatus intended for the acceptance of cultures is preferably made with an arrangement for the exposure of the resident culture to a gaseous medium. The said arrangement has a suction fitting with a suction opening for the intake of the gaseous medium and an exit opening, which is connected to a flow diversion for the guidance of the gaseous medium to flow immediately above the surface of the culture. For this purpose, the suction opening of the suction fitting exhibits a trumpet shaped inner surface widening in the direction of flow of the gaseous medium. In this case, analogous to the description made above, namely, that accompanying the trumpet shaped, continually widening suction opening, there is the advantage that dead zones which form themselves above the suction fitting are avoided, and from which, no fresh ambient air can be removed.

A construction kit for the assembly of a culture/exposure apparatus in accordance with a third aspect of the present invention includes separable upper and lower parts, the parts being separable to access a culture container. The lower part is configured to contain the culture container, and may include a supply unit for furnishing the cell culture in the culture container and a heater for temperature regulation of the liquid medium. The upper part supports an apparatus for the exposure of culture in the culture container to a gaseous medium. The exposure apparatus includes a suction fitting for the intake of the gaseous medium and a flow guide for the conductance of the gaseous medium to a plane above the surface of the culture.

Accordingly, a construction kit for the assembly of a culture/exposure apparatus is made, which contains four kit elements, namely, first, top parts separable from a culture container for access to the culture container, second, top parts with an apparatus for the exposure of the culture in a culture container to a gaseous medium, wherein the said exposure apparatus has as a third item, a suction fitting for the suction-removal of the gaseous medium and a flow directional means connected with the suction fitting for the guidance of the gaseous medium to the zone above the surface of the culture. The kit possesses further as a fourth item, a lower part element that accepts at least one culture container and may include a supply unit for furnishing the cell culture in the culture container and a heater for temperature regulation of the liquid medium. Thus, principally four different culture/exposure apparatuses can be assembled. These would be:
a first with an lower part element for the cultivation of cell cultures (or more generally of eukarotic cultures), which especially permits a submerse and basal nutrition with a feed liquid, together with a top piece with the invented flow guidance,
a second with the last mentioned lower part together with a “simplified” flow guidance (for example, with a through-penetrating, cylindrical flow duct)
a third with an lower part for the cultivation of prokaryotes, especially of bacterial in a Petri dish (without external supply with nutrient liquid), together with a top part with the invented flow regulation, as well as
a fourth with the last named lower part together with a top part with a “more simple” flow direction system.

A process for the culture and exposure of prokaryotes with the use of a culture/exposure apparatus with a recess for the reception of a culture container carrying the prokaryotes to be cultured as well as an apparatus for the exposure of the prokaryotes residing in the culture container to a gaseous medium in accordance with a fourth aspect of the present invention includes the exposure apparatus having a suction fitting for the intake of a gaseous medium and a flow duct connected with the suction fitting for the conducting of the gaseous medium to a plane above the resident prokaryotes in the culture container.

Accordingly, a procedure is given for the cultivation of prokaryotes with the aid of a culture/exposure apparatus with a receptor for the acceptance of a culture container holding the prokaryotes to be cultivated and also an apparatus is made for the exposure of the prokaryotes received in the said culture container to a gaseous medium. In this arrangement, the exposure apparatus comprises a suction fitting for the intake of the gaseous medium, a flow diversion means connected with the said suction fitting for the guidance of the gaseous medium over the surface of the prokaryotes residing in the culture container. In this operation, it is, in the first place, advantageous to create a culture/exposure apparatus, wherewith also prokaryotes (for instance, bacteria, fungi and the like) can be exposed to a predetermined gaseous medium, whereby their reaction thereto can be investigated. Up to now, such investigations were carried out only on eukaryotes, that is, mammalian cells. Prokaryotes, or special bacteria were cultivated and investigated only in connection with a liquid active material, for example cultivated when encapsulated in agar.

A culture/exposure apparatus for the carrying out of the above procedure in a fifth aspect of the present invention includes a recess for the reception of a culture container carrying the prokaryotes to be cultured as well as an apparatus for the exposure of the prokaryotes resident in the culture container to a gaseous medium. The exposure apparatus includes a suction fitting for the intake of the gaseous medium and a flow duct connected with the suction fitting for the conductance of the gaseous medium over the prokaryotes resident in the culture container.

Accordingly, a culture/exposure apparatus for the carrying out of a procedure described above with a recess for the receiving of a culture container with the prokaryotes to be cultivated as well as an apparatus for the exposure of the prokaryotes which are in the said container to a gaseous medium. When this is done, the exposure apparatus includes a suction fitting for the suction-removal of the gaseous medium and a flow guidance means connected with the said suction fitting for the guiding of the gaseous medium over the surface of the prokaryotes placed in the culture container.

The invention, as well as further features and advantages is now to be more closely described and explained with the aid of the attached drawing of embodiments.

### BRIEF DESCRIPTION OF THE DRAWINGS

FIG. 1 a schematic longitudinal cross-section through an invented culture/exposure device having the guidance of flow in accord with the invention,

FIG. 2 a schematic, profile view in section through an invented culture/exposure device having the guidance of flow in accord with the invention,

FIG. 3 a schematic plan view on the lower part of an invented culture/exposure device,

FIG. 4 (i.e., 4a to 4c) respectively, a sectional view, a perspective view angularly seen from above, a perspective view angularly seen from below of an invented flow duct,

FIG. 5 (i.e., 5a and 5b) respectively, a schematic top view and a profile view of an annular orifice in the invented culture/exposure device for the uniform apportionment of the suction flow about the guided flow,

FIG. 6 (i.e., 6a and 6b) respectively, a profile view and a plan view of a flow-spin body which is placed in the invented flow guidance,

FIG. 7 a schematic sectional view through the placement of an invented flow duct and a culture container with a culture placed therein,

FIG. 8 (i.e., 8a and 8b) respectively, a schematic sectional view of an invented suction fitting in accord with a first embodiment and a schematic plan view of a bottom zone of this invented suction fitting,

FIG. 9 (i.e., 9a and 9b) respectively, a schematic sectional view of an invented suction fitting in accord with a second embodiment and a schematic plan view of a bottom zone of this invented suction fitting,

FIG. 10 a perspective, angular view of an additional upper component for the invented kit for the assembly of an invented culture/exposure device,

FIG. 11 a perspective profile view of an additional lower part for the acceptance of cell culture containers with a supply apparatus for delivering to the cell cultures a fluid medium, which said apparatus is a component of the invented kit for the assembly of an invented culture/exposure device, and

FIG. 12 a perspective profile view of an invented culture/exposure device assembled from the upper component of FIG. 10 and the lower part of FIG. 11.

### DETAILED DESCRIPTION OF THE INVENTION

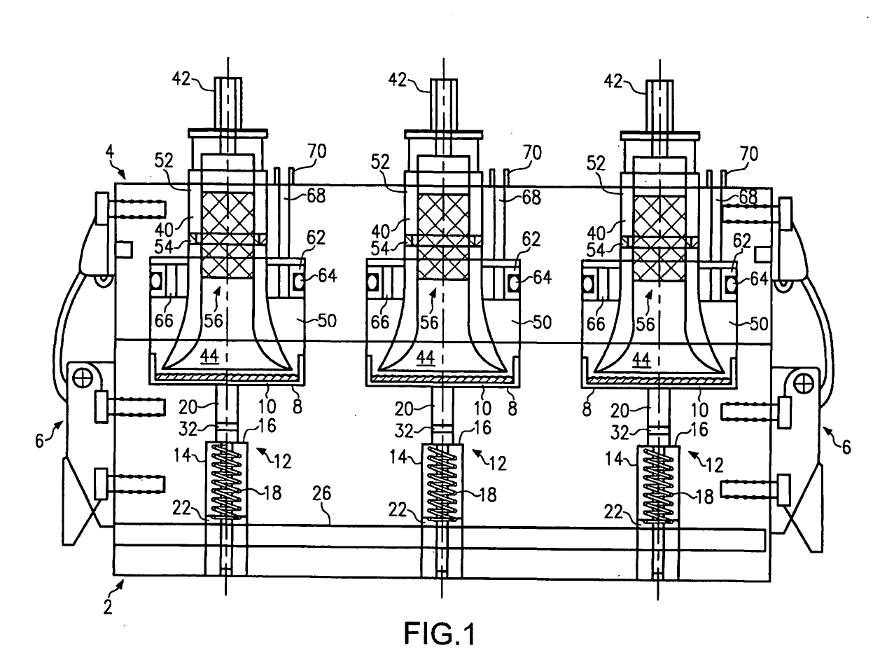

FIG. 1 shows a schematic, longitudinal cross-section through an invented culture/exposure apparatus. The said apparatus is made in a nearly boxlike form and consists, accordingly, of two halves, one superimposed on the other, namely an assembly of an lower part 2 and an upper part 4. In the combined condition, lower part 2 and upper part 4 can be held together by a fastener 6 in the form of a latch. The lower part 2 contains, in this example shown in FIG. 1, three recesses 8 formed to receive three culture containers 10. Obviously, this number can be optionally selected. The recesses 8 are cylindrical borings in the blocklike lower part 2, as may be more clearly seen in the top view of FIG. 3. Also, the culture containers 10 have the form of a circular, open container, wherein the outer walls are obviously cylindrical. The height of said walls of the containers 10 is somewhat lower than the depth of the said cylindrical recesses 8.

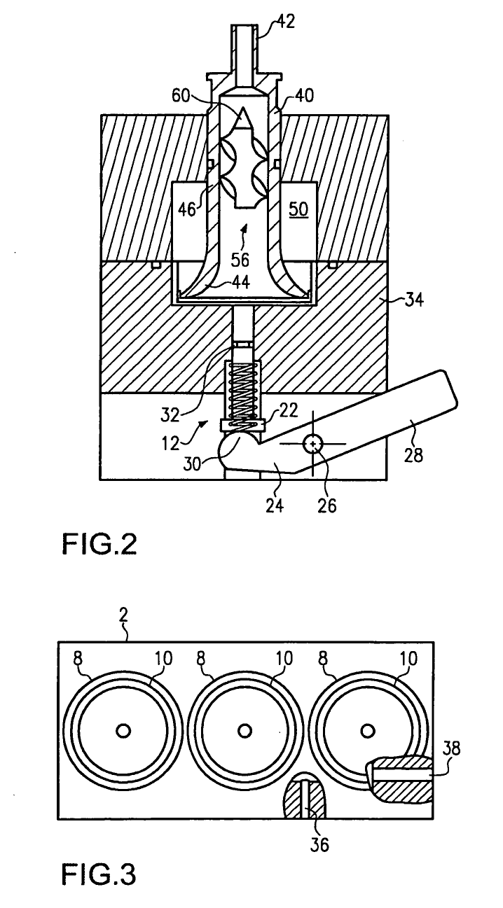

An ejector mechanism 12 is provided for the removal of the culture containers 10, which is shown with particular clarity in the section view of FIG. 2. The ejector 12 includes a cylindrical penetrative boring 14, which extends itself from the bottom of the lower part 2 up into the recess 8. As seen from the said bottom of the lower part 2, this boring 14 is made with an offset 16 which provides an abutment for a return spring 18. Guided for back and forth movement within this boring 14 is an ejector pin 20 and the outside diameter of said pin is compatible with the smaller inside diameter of the boring 14, upward from the offset 16. The ejector pin 20 has a cylindrical head 22, the outside diameter of which slidingly corresponds to inside diameter of the penetrative boring 14 in its larger section. In this way, an exact guidance of the ejector pin 20 in the penetrative boring 14 is assured. On the underside of the said head 22 is attached the return spring 18. Thus the return spring 18 prestresses the ejector pin 20 in a direction away from the recess 8. On the flat, outer side of the head 22 is to be found attached a lever 24, the active end of which pivots in the longitudinal direction of the boring 14 about an axis of rotation 26, slightly to the side of the boring 14. The opposite end of this lever 24 protrudes from the underside of the lower part 2 in the form of a manually activated handle 28 and so accessibly extends out of the culture/exposure apparatus. As may be seen in FIG. 1, for all three ejection mechanisms 12, one, common pivot axle 26 in the form of continuous shaft is provided to extend itself completely through the lower part 2. This pivot axle 26 is motionlessly fixed in the lower part 2, while the lever 24 can freely rotate about it. Thus there can be found, extending out of the longitudinal side of the lower part 2 three handles 28, with which the three culture containers 10 may be individually ejected by hand. The lever 24, in detail, possesses a rounded activation cam 30, which raisingly contacts the open end face of the head 22. This activation cam 30 allows the pivotal movement of the lever 24 to be carried out in the most friction-free manner possible for the reciprocal movement of the ejector pin 20. The ejector pin 20 meets, in the upper, smaller diameter part of the boring 14 an annular seal 32, which closes off the recess 8 from the lower section of the ejector mechanism 12. Obviously, any other kind of ejection mechanism is possible, with which the culture container 10 can be more easily removed from its recess 8.

The lower part 2 is, essentially, hollow, and forms around the discussed recess 8 a liquid-tight chamber 34 which is filled with a liquid for the temperature regulation of the recess 8 and thereby also provides temperature regulation of the culture which is in the culture container 10. For temperature control on the chamber 34 can be found a liquid inlet 36 and a corresponding liquid outlet 38 (clearly seen in FIG. 3). These fittings 36, 38 can be connected to an external heating circulation for the adjustment of the temperature in the chamber 34. It is obviously possible that alternative heating apparatus can encompass the recess 8, for instance, a heating coil or the like, with which the culture container 10 can be kept at a predetermined temperature.

The top-part 4 encompasses an exposure apparatus for the subjection of the culture in the culture container 10 to a gaseous medium. The gaseous medium can be, for example, a pure gas. That is to say, all the therein contained substances such as atoms, molecules and the like find themselves in the gaseous phase. The gaseous medium can also be a carrier for entrained solids and/or liquid particulate, or again may be a mixture of the above, for the purpose of bringing a gaseous medium into contact with the culture. Further it is possible that the gaseous medium may be or may carry such substances as aerosols, atomized liquids, small droplets, or plant-protection means such as spray fogs, Brownian size particulate, solid particulate such as wood dusts, or colloidal suspensions in gas, or atomized suspensions or yet emulsions. For example, the loading of lung cells with cigarette particulate can be investigated in this suspended form. The here numbered materials are not conclusive, but may further vary in accord with the investigations at hand.

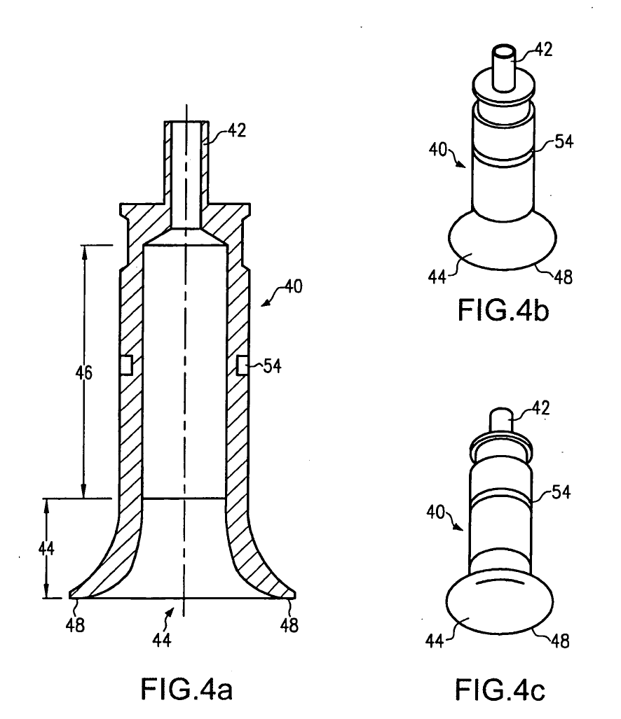

The exposure apparatus encompasses a flow duct 40 with an entry 42 in the form of a connection fitting and an exit fitting 44, which opens closely above the surface of the culture in the culture container 10 located in the lower part 2. The flow duct 40 includes a cylindrical transition section 46 with a cylindrical inner boring, which blends continuously in the flow direction into the widening trumpet opening inner surface of the exit opening 44. This inner surface is, in the flow direction, preferably hyperboloid in shape (see, in particular, FIGS. 4a-4c). The outer shape of the flow duct 40 is independent of the hyperboloid inner surface. In accord with the application (composition of the aerosols, etc.) it is possible that various shapes of the opening edges 48 of the exit opening 44 can be advantageous (for instance, rounding off, edges being as sharp as possible and the like). The inner surface of the outlet opening 44 runs onto the outlet rim 48 exactly in a horizontal plane. That is to say, the hyperbolic shape of the section inner surface is so designed, that it possesses a curving to the horizontal. All together, the hyperbolic curve generates itself from the completely vertical run of the flow guide section 46 (FIG. 2) and curves into the present fully, horizontal direction at the outlet edge 48. This transition of flow direction leads to a flow turning a 90° angle radially away from the central axis. This special geometry makes possible a smooth, turbulence-free outward flow, which, among other things, assures the continual feed of fresh, gaseous medium onto the culture surface. Moreover, the circumferential, concentration apportionment of the medium onto the surface to be treated is nearly completely uniform.

The flow duct 40 is frictionally held in a penetrative boring 52 in the upper part 4, which boring extends from the top side thereof and opens into an inner chamber 50. In the outside surface of the cylindrical guide section 46 is a is a ring shaped groove 54 designed to accept a complementary ring sealing means, such as an O-ring. This ring shaped sealing means now between the outside wall of the flow duct 40 and the inner wall of the through boring 52 seals off, in an airtight manner, the outer space of the culture/exposure apparatus from the inner chamber 50. The flow duct 40 is slidably and longitudinally placed in the through boring 50, whereby the distance of the opening edge 48 to the surface of the culture in the culture container 10 can be adjusted. For information in this regard, see the more detailed discussion below.

The inner chamber 50 forms, in the assembled condition of the lower part 2 and the upper part 4 with the recess 4, a closed inner space, cylindrical in shape, wherein the inner chamber 50 and the recess 8 fit into one another without edge impact damage.

In the cylindrical guidance section 46 is inset a twist body 56, which is held by frictional closure. For details of the twist body 56, refer to FIGS. 6a, 6b. The twist body 56 is a short cylindrically shaped piece, the outer diameter of which corresponds to the inner diameter of the cylindrical section of the guidance device 40 into which three neighboringly placed, spiral shaped blades 58 are incised. Obviously, the number of the neighboring blades in relation to the length of the entire twist body is of such a nature, that essentially, they follow a path through half a full rotation about the cylinder shaped twist body 56. Further, the blades 58 are placed so close to one another, that the remaining web which lies therebetween is as thin as possible. In addition, the through-flow cross-section openings between all blades 58 are as large as possible. Centrally, on the upper end of the twist body 56 is formed a conical apex 60, the external wall of which makes a transition at its base in an “impact-free” manner into the bottom of the blades 58. The conical apex 60 is directed counter to the direction of the flow. In this connection, the concept “impact-free” is so selected, that indeed, if necessary, a more or less sharp bend is present between the outside surface of the conical apex 60 and the inner surface of the blades 58 at their deepest position. However, in this is no sharp offset, which otherwise might engender turbulent eddies which could be imparted to the flow. In FIG. 6b the circular base surface of the conical apex 60 is shown in dotted lines.

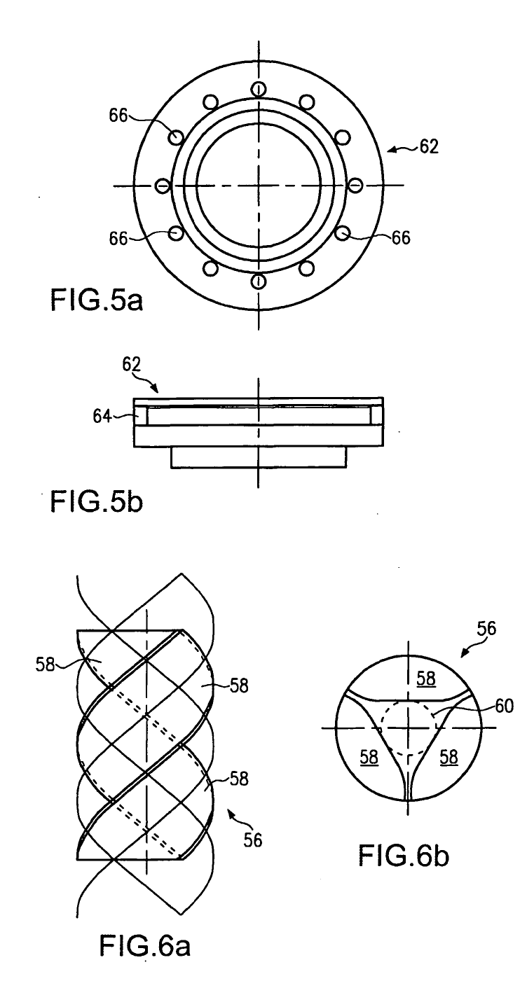

Around the cylindrical guiding section 46 of the flow duct 40, additionally, a disk like, annular orifice 62 is placed, which is slidable in the longitudinal direction. The inner diameter of this said annular orifice 62 corresponds to the outer diameter of the cylindrical guidance section 46 and the outer diameter of the said annular orifice 62 corresponds to inner diameter of the inside chamber 50 of the upper part 4.(See FIG. 1.) On the cylindrically shaped outer wall of the annular orifice 62 is, further, a ring groove 64 into which a sealing ring can be inserted, which seals off the cylindrical outer wall of the annular orifice 62 against the inner wall of the inner chamber 50. As may be inferred from FIG. 5a, the annular orifice 62 encompasses several axial running through-borings 66, which allow communication between the upper space section of the inner space 50 with the lower space section of the same although the said sections are separated by the intervening annular orifice 62. On the upper side of the inner space 50 is also a boring 68 up to a connection fitting 70, onto which, for example, a hose may be attached and connected to a vacuum pump. In this way, the inner space 50 is placed under suction. For example, it is possible for one vacuum pump to serve all three of the culture/exposure apparatuses shown in FIG. 1 by means of connection with a common, appropriately subdivided hose. The annular orifice 62 serves also the uniform rotation-symmetric apportionment of the suction and the thereby induced flow about the entire outer surface of the flow duct 40 in order to compensate for the lack of rotational-symmetry from the duct 40 because of the placement of the through borings 66 (see also FIGS. 5a, 5b). Beyond this, it is possible that various annular orifices 62 per flow duct 40 can be provided, which differ among themselves in the dimensioning of their penetrative boring 66. In each case, in accord with the adjustment to be made in the suction in the flow dust 40, an appropriate annular orifice 62 is selected and slipped over the flow duct 40.

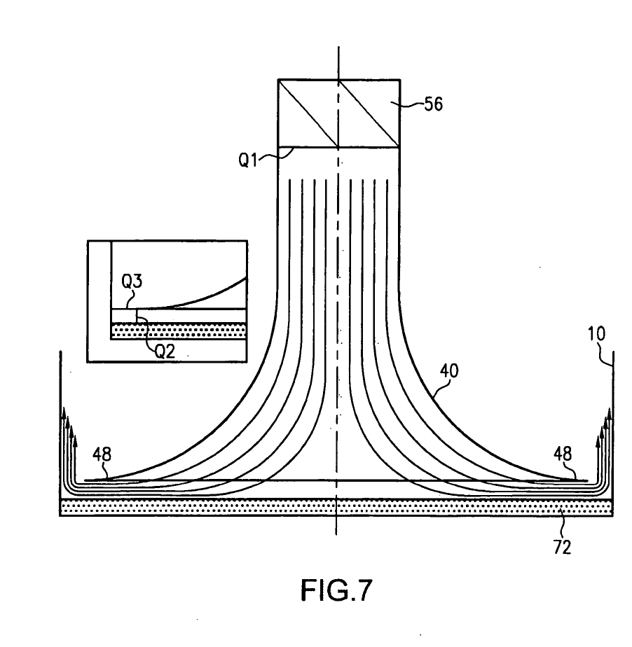

FIG. 7 shows a schematic sectional view through the flow duct 40 and the culture container 10 for the explanation of the adjustment of the separating distance between the opening 48 and the surface of a culture 72 held in the said culture container 40. The separating distance of the outflow opening 44 to the surface of the culture 72 is so adjusted, that the through-flow cross-section Q2 of the annular orifice between the opening edge 48 and the surface of the culture 72 is less than the through-flow cross-section Q1 in the cylindrical guidance section 46 of the flow duct 40. With this measure, the flow over the surface of the culture 72 accelerates, which once again avoids the formation of turbulent eddies. As a rule, the separating distance of the outlet rim 48 from the surface of the culture 72 lies in the millimeter range, for example, perhaps only 1 mm. Such a minimal separating distance is also required on the basis, that quite likely the surface of the culture 72 can be uneven in some places. As an additional measure, provision has been made that the ratio of the inner diameter of the container 10 and the outer diameter of the outlet opening 44 on the opening rim 48 is to be so dimensioned, that the flow cross-section Q3 of the ring opening between opening rim 48 and the inner wall of the container 10 is likewise greater than the previously named through-flow cross-section Q1. Generally, no restrictions are imposed on the height of the flow duct 40—exclusive of the design of the hyperboloidic shape of the outflow opening 44. Obviously, the cylindrical guidance section 46 may indeed be omitted and the entrance can introduce the flow directly into the hyperboloidal curvature 44. The diameter of the outflow opening 44, measured across the rim 48, is, in any case, determined by adhering to the stated ratio Q3/Q1 with consideration given to the diameter of the culture container 10, which can be the standard container which is most used in practice.

The separating distance of the outflow edges 48 from the surface of the culture 72 can be adjusted in multiple ways. First, in the recess 8, before the inset of the culture container 10 containing the culture 72, an adjustment platelet of known thickness may be laid therebetween. Subsequently, the flow duct 40, upon the closing of the lower part 2 and the upper part 4, may be pushed downward, just so far against the frictional holding force, until the outflow rim 48 impacts the adjustment platelet. Subsequently, the culture/exposure apparatus can be opened, the said adjustment platelet be removed, and in its place the culture container 10 with its resident culture 72 may be inserted. Second, as an alternative to the above, in the top area of the upper part 4, an adjustment apparatus for the manual or automatic displacing of the said separating distance is provided. The manual adjustment apparatus can, for example, be a spiral drive with a worm gear serving as the manual activation agent. In this case, the spiral drive acts between the upper part 4 and the flow duct 40. In yet another adjustment arrangement, it is possible that graduation markings may be inscribed on the end of the flow dust 4 extending from the upper part 4, with which markings one or more selected separating distances may be emphasized. Instead of the frictional binding between the flow duct 40 and the upper part 4, it may also be advisable to employ a screw fitting, so that the said separating distance may be adjusted by rotating the flow duct 40 to achieve a threaded advance or retraction thereof relative to the upper part 4.

For the temperature control and conditioning of the gaseous medium, it is advisable to place a heating coil about the flow duct 40, which would be connected to a corresponding heating source for the adjustment of the temperature. Alternatively thereto, the flow duct 40 can be constructed of a corrosion resistant metal (titanium could be recommended) or at least be encased within said metal, and a current be directed to and through the corrosion resistant metal, which would result in the warming thereof.

In addition, it is possible that either between the entry 42 of the flow duct 40 and a therewith bound suction fitting or between the suction fitting 70 and the vacuum pump a flow measuring device based on volume per time unit or on a mass sensitive element with an attendant control valve could be placed in the circuit. In this way for example, the through-flow quantity of the gaseous medium through the said flow duct 40 can be specifically controlled.

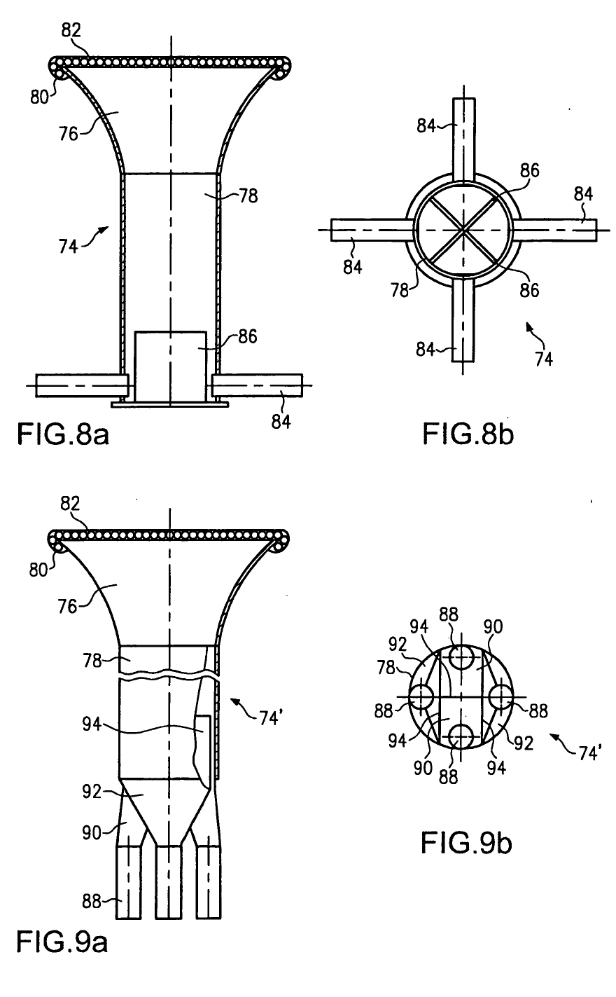

In the FIGS. 8a, 8b, 9a, 9b are shown two alternative designs of invented suction intake fittings 74; 74′. These said fitting can be connected with the inlet 42 of the multiple flow dusts 40. The suction fittings 74; 74′ are constructed, in principle, in a similar manner to the flow duct 40. That is, these embrace a suction opening 76 with a trumpet shaped inner surface directed counter to the direction of flow. This inner surface possesses, again, in the flow direction, a hyperboloid design. Further, there is connected to this inner surface a cylindrical guidance section 78 with a cylindrical inner surface. The suction opening 76 in, as said, hyperboloid shape, can—otherwise than is indicated in the FIGS. 8a, 9a—advantageously open themselves to the extent, that the generally circumferential edge 80 resides in a horizontal plane. Thereby, once again a flow diversion of the flow present at the suction opening 76 occurs to the extent of approximately 90° away from the horizontal and into the vertical path. In regard to the opening of the suction fitting 76, is overlaid with a large porosity, foamed material 82. This foamed material overlay assures a uniform flow and damping within the suction intake fitting 75 itself as a protection against erratic movement and turbulence in the source of ambient air. Nevertheless, the passage of smoke or other solid particulate is not hindered nor filtered out. In this way, prevention is especially extended against lateral currents which strike internally on the suction fitting 74; 74′ and disturb the uniform distribution of the effective flow within the suction opening 75. Instead of the said overlaid foamed material 82, it is also possible that a large opening screening be provided in the suction opening 76. Alternately also, use may be made of another material which damps cross flows in the suction opening 76.

The suction fitting 74 shown in FIGS. 8a, 8b possesses, in turn, connection fittings 84, which are directed radially outward. These fittings 84 are connected in the bottom zone of the cylindrical duct section 78 and serve for the connection of lines to the individual entry fittings 42 of the flow ducts 40. As may be seen in FIG. 8, the said, connection fittings 84, which are radially projecting from the duct section 78, are placed to be rotationally symmetric about the outer wall thereof. In the illustrated example, four connection fittings 84 are provided, offset at 90° from one another. Obviously, it is allowable, that another number of such connection fittings 84 be chosen, for instance eight thereof. In order to avoid the switching of flows among the four oppositely set connection fittings 84 within the cylindrical duct 78, therewithin are to be found symmetrically disposed guide vanes 86 in the bottom of duct 78. These reach from the closed bottom of the said duct 78 approximately twice as high as the plane of the openings of 84, which same are equally high and near the bottom of duct 78. In this way, the four partitions 86 form four quarter-circular chambers. The ratio of the length to the diameter of the duct section 78 lies approximately at 2, whereby the length of the straight line, radial projecting connection fittings 84 more or less correspond to the diameter of the duct 78. The diameter of the said connection fittings 84 is approximately ten times smaller than the length of the guide duct 78 and the height of the said partitions 86 runs in effect, some three time the diameter of the connection fitting 84.

In 9a, 9b is to be seen an alternative embodiment of the connection fittings 74′, wherein said fitting possesses four connection fittings 88 projecting axially parallel from the bottom of the guide duct 78. These four connection fitting 88 enter through appropriately shaped transition sections 90, 92 into the cylindrical interior of guide duct 78. As is evident from FIGS. 9a, 9b, the transition sections 90, 92 are not designed to be rotationally symmetric to the connection fittings 88 although this is contrary to the positioning of the connection fittings 88 themselves in reference to the bottom of the guide duct 78 On this account the corresponding separation walls 94 are provided for the apportionment of the flow into equal parts through the guide duct 78 respectively to the four connection fittings 88. These walls embrace two separation walls 94 which divide a horizontal (as per FIG. 9b) centerline between the two transition sections 92 into three approximately equal lengths. Further, in this way the said separation walls 94 stand vertically to the said center line as does a third separation wall 94 encompassing the said center line. This third separation wall is placed between the two first named separation walls 94 and divides the transition section 90 within the cylindrical guide duct 80 into two separate parts. In regard to this suction fitting 74′, the ratio of the height to the diameter of the guide duct 78 is about 5, the ratio of the diameter of the guide duct 78 to the suction fittings 88 is about 4 an the ration of the height of the separation walls 94 to the height of guide duct is about 7. The three separation walls 94 are equal in height.

Obviously, such a suction fitting 74 can also attach itself directly onto a flow duct 40. That is to say, do so without the connection by means of its connection fittings 84, the connection line and the entry 42 of the flow duct 40. The cylindrical guide duct 78 of the suction fitting 74 can thus communicate directly with the same diameter into the cylindrical flow section 46 of the flow duct 40 or it can even place the trumpet shaped suction opening 76 of the suction fitting 74 directly into the trumpet shaped opening of the outflow opening 44 of the flow duct 40. The suction fittings 74; 74′ can either be positioned in the ambient atmospheric air or in a corresponding space, into which space the entrained particulate from the gaseous medium is artificially injected. That is to say, for example, liquid droplets, by means of appropriate spray nozzles, were atomized into the aid space or again solid materials were blown in through an opening in the said space, or yet a smoking robot could produce smoke in the said room, along with other possibilities.

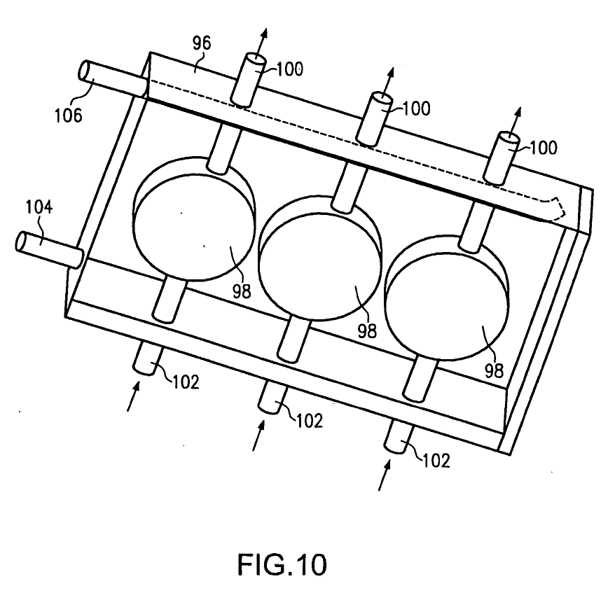

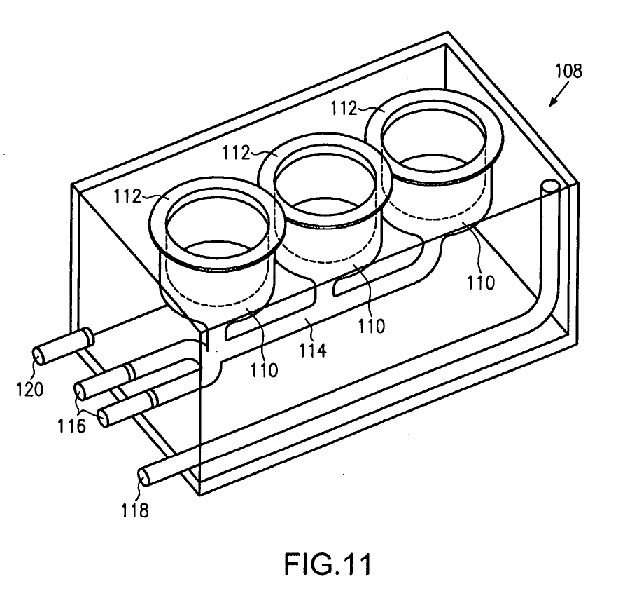

In the following, further components in the form of upper and lower parts of an assembled kit for a culture/exposure apparatus are described, which in combination with the above described upper part 2 and lower part 4 can be combined into the construction kit. In this way, FIG. 10 shows a top part 96 with a simplified exposure apparatus for the treatment of cultures with a gaseous medium. This top part 96 then encompasses, as per FIG. 10 two major components, namely a top part 96 with a simplified Exposure apparatus for the treatment of culture with a gaseous medium and three essentially container-like recesses with a rotation symmetrical inner surface for the guidance of the gaseous medium up to the surface of the culture. The flow guidance means 98 possess at the height of their opening rims a cylindrical inner profile which transforms into a flat bottom structure. Penetrating from the two longitudinal sides of the upper part 96 into the flow guides 98 are connection fittings 100 and 102, which are serving as feed and removal conduits for the gaseous medium. The gaseous medium moves, for example, in this way by means of feed functioning connection fittings 102 (which, for example, is bound with the invented suction fittings 74; 74′ at a side wall into the flow guide 98. The said gaseous medium is removed therefrom by induced suction through the suction connections 100, which are functioning as exit means for the flow. The said induced suction can be generated, for example, by connection to a vacuum pump.

In this case, when seen from a flow-technology standpoint, the matter concerns a simply designed flow guidance means 98, which does not possess a uniform time and space, homogenous apportionment of the gaseous medium over the entire surface of its outflow opening. Further, the upper part 96, designed as a liquid tight hollow body possesses also a feed connection 104 and a removal connection 106, both intended for a hot liquid with which the flow guidance means 98, and therewith the gaseous medium can be temperature controlled over the culture.

FIG. 11 shows an additional lower part 108, which is constructed for receiving culture containers with the cell culture contained therewithin. The cell cultures may be in general, eukaryotes. To this end, the aid lower part 108 possesses three recesses 110 to receive three culture containers 112 (for example, Transwell-inserts) in which the cell culture may be placed. At this point, reference is made to German Patent 198 017 63, which has disclosed an apparatus for cell cultivation. The disclosure of this patent is herewith fully accepted in the present application.

These culture holding containers 112 (Transwell-inserts) possess, for example, a cup-like shape with a circular cross-section, wherein the diameter of the container opening to the container bottom is diminishly conically shaped. The container bottom consists of a porous plastic material, that is, of polyethylene phthalate. The cell culture-insert offers a liquid permeable carrying structure for a membrane, which, in accord with the current requirement of the cells to be cultivated, can be made from various plastic materials, for instance, from the said polyethylene phthalate. In this operation, the membrane carries the cell culture.

The recesses 112, in their bottom areas, are bound with a common line system 114. This line system in turn, branches into two connection fittings 116, to which a liquid level controller can be attached with which the cell cultures in the culture containers 112 can be supplied with liquid nutrient substances. For example, a pulse-like control of the feed and removal thereof can be included. The controller, which not described here in greater detail, controls the level of the liquid medium within the culture container 112. Therewith the cell cultures within the culture container 112 can be periodically nourished in a basal and submersible manner, since, correspondingly, the liquid level of the liquid nutrient can be introduced above or beneath the surface of the cell culture. For further details in regard to the pulse control and level regulation of the liquid medium within the culture container 112, reference should me made to the above cited patent application. The lower part 108 is designed, once again as a fluid tight hollow body, with a liquid feed connection 118 and a liquid outlet connection 120. Through these connections a temperature controlled liquid can be conducted through the lower part 108 for temperature regulation of the cultures held in the culture container 112. The receptacles, i.e. the culture container 112 are likewise liquid tight sealed against the said inner space.

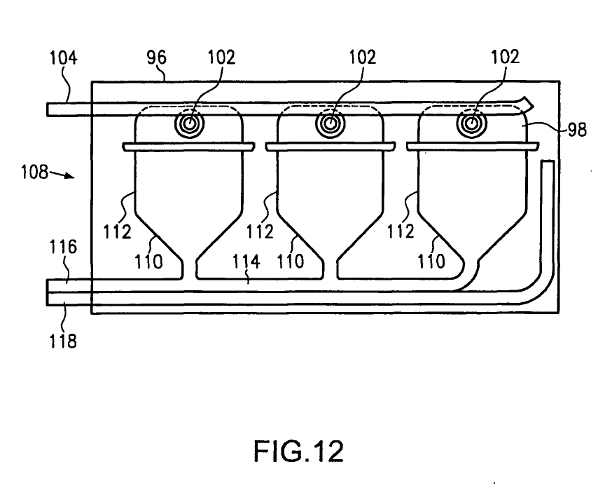

FIG. 12 shows a variant of a culture/exposure apparatus which can be assembled, wherein the lower part 108 of the FIG. 11 is combined with the upper part 96 of FIG. 10. The combined kit is so designed, that even the lower part 2 and the upper part 4 can correspondingly be combined with the lower or under part 108 and the upper part 96, in accordance with which culture is to be investigated. For example, for cell cultures the under part 108 would be used, for prokaryote-cultures, under part 2 is recommended, whereby these cultures, then, for the purpose of nourishment in an appropriate substance, for instance, agar, can be held in the culture containers. The said arrangement of upper and lower parts is to be recommended if homogeneity of the most possible timely and spatial characteristics is desired in the apportionment of the gaseous medium to which a culture is to be exposed. In a case wherein this is critical to an investigation, then the upper part 4, along with the invented flow guidance features should be used, if this is less critical to an investigation, and the upper part 98 would be recommended therewith.

All together, the culture/exposure apparatus can be so designed, that it can be operated following the equipping of an automatic line by robots with corresponding culture containers, 10 or 112 and provided with the contained cultures to be contained therein (these being, for example, eukaryotes or prokaryote cultures). In this case, however, instead of the clasping mechanism 6 as shown in FIG. 1, a robot operable closure can be provided. If this is done, then the lower part 2, 108 and the upper part 4, 96 can be easily opened and closed by a robot. Further, on the culture containers 10; 112, for example, means can be provided such as magnets, indentations, frictional protuberances and the like, with which a robot arm can easily seize the culture container 8, 110 and remove same, and of course, perform the converse operation of replacement. Finally, the culture/exposure apparatus as a whole is so designed, that in a cleaning station, all culture containers 10; 112, after they have been removed, can be easily washed. Another advantage is, that as far as possible, edges and other difficultly accessible places, which come into contact with the gaseous medium, have been avoided.

Especially, in accord with the invention, with the culture/exposure apparatus as shown in FIG. 1 (wherein the upper part 4 can be changed to the upper part 96, if a corresponding homogenous apportionment of the flow is not necessary), prokaryotes (that is, for example, bacteria, fungi, etc.) can be cultivated and subjected to a gaseous medium, which opens a fully novel possibility for investigation with this kind of cultures. Up to now, such investigations were carried out only on cell cultures. Thus it is now possible to carry out the Ames-test, which up to now could only be executed with liquid starting materials (which were encapsulated with the bacteria in agar). This can now be done with the active material carried in the gaseous medium (or with the gaseous medium itself, or with the therein contained liquid or solid entrainment).

The said investigations can now be extended, since the bacteria were, under certain circumstances, only encapsulated with their nutrient material in agar and the active material flowed over the encapsulated bacteria.

The flow of the gaseous medium intended for cultivation treatment would be, in such investigations, normally adjusted to the following values: about 80 ml/minute, about 50 ml/minute, about 10 ml/minute, so that the flow within the flow-guidance means lies in the lower ranges of the Reynold numbers and can be classified as linear, non-turbulent flow. Further, in the case of investigations of the effects of tobacco smoke on lung cells, smoke/air mix ratios of 1 to 5 up to 1 to 10 have been adjusted, and this smoke/air mix was held at a temperature of some 35° C. (for example by means of the corresponding heating means about the flow guide ducts 40, 98). Of the smoke which is carried in the air, in accord with laboratory determinations, about 1% was bound by the lung cells.

1-56. (canceled)

57. An apparatus for exposing a culture having a surface to a gaseous medium, the apparatus comprising:
a housing configured to house a culture within the housing;
a flow duct to direct a flow of gaseous medium to a culture in the housing; and
the flow duct comprising an outflow opening disposed above the surface of the culture when a culture is in the housing, the outflow opening comprising an inner surface opening in a trumpetlike configuration in the direction of flow.

58. The apparatus of claim 57 wherein the inner surface is hyperboloid-shaped and is rotationally symmetrical about an axis.

59. The apparatus of claim 57 wherein the outflow opening comprises a minimum cross-section area and an outer rim, the rim spaced from the surface of the culture when a culture is in the housing; and
the rim and the surface of the culture define an annular flow passage therebetween, the flow passage having a cross-section area less than the minimum cross-section area of the outflow opening.

60. The apparatus of claim 57 wherein the outflow opening is configured to be spaced a separation distance from the surface of the culture when a culture is in the housing and the apparatus comprises means for selectively adjusting the separation distance.

61. The apparatus of claim 57 wherein the housing comprises a wall enclosing a recess to receive a culture container having an inner side wall;
the outflow opening comprises a minimum cross section area and an outer rim in the recess; and
the inner side wall and the rim define an annular gap therebetween, the gap having a cross-section flow area greater than the minimum cross section area of the outflow opening.

62. The apparatus of claim 57 wherein the housing comprises a chamber to house a culture;
the flow duct discharges into the chamber to flow gaseous medium to a culture housed in the chamber; and
the housing comprises a vacuum line and a vacuum port, the vacuum line extending from the chamber to the vacuum port, the vacuum port being configured to be fluidly connected to a source of vacuum whereby gaseous medium discharged from the flow duct into the chamber flows out of the chamber through the vacuum line.

63. The apparatus of claim 62 wherein the housing comprises a bore wall and an annular orifice, the bore wall defining a bore extending into the housing to the chamber, the flow duct at least partially in the bore, the bore and the chamber extending along a common axis;
the annular orifice in the bore surrounding the flow duct, the annular orifice forming a substantially airtight seal between the flow duct and the bore wall and dividing the bore into upstream and downstream portions; and
the vacuum line comprises a plurality of passages through the annular orifice fluidly communicating the upstream and downstream bore portions, the passages being arranged symmetrically about the axis.

64. The apparatus of claim 63 provided with a plurality of annular orifices, each annular orifice having a different cross-section passage area, whereby suction generated through the vacuum line can be selectively adjusted by selectively inserting one of the plurality of different annular orifices into the apparatus.

65. The apparatus of claim 57 comprising at least one of the following (a), (b), (c) and (d):
(a) means for measuring the flow rate of the gaseous medium;
(b) means for regulating the temperature of gaseous medium discharged from the flow duct;
(c) a heating device in the flow duct to regulate the temperature of gaseous medium discharged from the flow duct; and
(d) a flow meter configured to measure the flow rate of the gaseous medium, a flow rate adjusting valve operatively connected to the flow duct to selectively adjust the flow rate of the gaseous medium through the flow duct, and a connection between the flow meter and the flow rate adjusting valve wherein the flow rate adjusted by the flow rate adjusting valve is a function of the flow rate measured by the flow meter.

66. The apparatus of claim 57 wherein the housing comprises a plurality of chambers, each chamber configured to house a respective culture container;
the flow duct comprises a plurality of flow ducts, each flow duct associated with a respective chamber to flow gaseous medium to a container housed in the chamber; and
the apparatus comprises a suction fitting and a plurality of flow lines, the suction fitting configured to be attached to a supply of the gaseous medium, each flow line extending from the suction fitting to a respective chamber, whereby the supply of gaseous medium is fluidly connected to each chamber.

67. The apparatus of claim 57 wherein the housing comprises an upper part, a lower part, and a connection separably joining the upper and lower parts;
the flow duct is mounted in the upper part; and
the lower part comprises a recess to house a culture and means for regulating the temperature of a culture housed in the recess, the recess having an open end when the lower part is separated from the upper part whereby access to a culture is achieved by separating the upper and lower parts.

68. The apparatus of claim 67 wherein the lower part comprises means for furnishing a liquid medium to a cell culture housed in the recess.

69. The apparatus of claim 68 wherein the means for furnishing a liquid medium comprises at least one the following (a) and (b):
(a) means for submersible or basal supply of the liquid medium; and
(b) means for regulating the temperature of the liquid medium.

70. The apparatus of claim 67 wherein the lower part recess is configured to house a container holding a prokaryotic culture; and
the apparatus comprises an ejection apparatus attached to the lower part, the ejection apparatus being actuatable to engage a container in the recess to eject the container from the apparatus.

71. An apparatus for exposing a culture having a surface to a gaseous medium, the apparatus comprising:
a housing configured to house a culture within the housing;
an apparatus for flowing gaseous medium into the housing;
a flow line in the housing to flow gaseous medium to the culture;
the flow line comprising a suction opening to receive the gaseous medium from a supply of the gaseous medium and an outflow opening disposed above the surface of the culture when a culture is in the housing;
the suction opening comprising an inner surface opening in a trumpetlike configuration opposite the direction of flow through the flow line.

72. The apparatus of claim 71 wherein the inner surface is hyperboloid-shaped and is rotationally symmetric about an axis.

73. The apparatus of claim 71 wherein the suction opening is covered by a gas-permeable cover formed from a foamed material of large porosity.

74. The apparatus of claim 71 wherein the flow line comprises a plurality of outlet openings and a plurality of branch lines, the branch lines fluidly connecting the suction opening and the plurality of outlet openings whereby flow of gaseous medium through the flow line divides into a plurality of flow paths.

75. The apparatus of claim 74 wherein the flow line comprises a guide section between the suction opening and the plurality of branch lines, the guide section comprising a cylindrical inner surface extending along an axis, and each branch line extends radially away from the guide section.

76. The apparatus of claim 74 wherein the flow line comprises a guide section between the suction opening and the plurality of branch lines, the guide section comprising a cylindrical inner surface extending along a first axis, and each branch line extends along a respective second axis substantially parallel with the first axis.

77. The apparatus of claim 74 wherein the flow line comprises a guide section and a plurality of guide vanes in the guide section;
the guide section between the suction opening and the plurality of branch lines, the vanes arranged to separate suction spaces in the guide section, each suction space fluidly connected with a respective branch line, wherein the guide vanes are configured to generate substantially equal suction in each suction space.

78. A construction kit for the assembly of at least one of a cell culture/exposure apparatus and a prokaryotic culture/exposure apparatus for exposing a culture to a gaseous medium, the kit capable of constructing a plurality of different culture/exposure apparatus, the construction kit comprising:
at least one of first and second upper parts; and
at least one of first and second lower parts;
each upper part configured to cooperate with each lower part to form a culture/exposure apparatus by selecting one lower part and one upper part;
each lower part comprising an upper side and a recess open on the upper side, the recess configured to receive a culture;
the first lower part being configured to house a prokaryotic culture;
the second lower part being configured to house a cell culture, and comprising means for supplying nutrients to the cell culture in the recess;
each upper part comprising an upper side, a lower side that faces the upper side of the one lower part when forming a culture/exposure apparatus, and a flow duct to direct a flow of a gaseous medium to a culture, the flow duct comprising an outflow opening on the lower side that opens into the recess of the one lower part when forming a culture/exposure apparatus;
the outflow opening of the first upper part comprising an inner surface opening in a trumpetlike configuration in the direction of flow.

79. A method for the culture and exposure of prokaryotes comprising the steps of:
(a) providing a housing comprising a chamber, an inlet, an outlet, a first flow line fluidly connecting the inlet and the chamber to flow gaseous medium into the chamber, and a second flow line fluidly connecting the outlet and the chamber;
(b) placing a culture of prokaryotes into the chamber;
(c) connecting the inlet to a source of gaseous medium; and
(d) connecting the outlet to a source of vacuum,
whereby the vacuum is fluidly connected to the inlet and induces flow of gaseous medium through the chamber to expose the culture of prokaryotes to the flow of gaseous medium.

80. A culture/exposure apparatus for exposing prokaryotes to a gaseous medium, the apparatus comprising a housing, the housing comprising a chamber to house a culture of prokaryotes, a first flow duct in the housing to be connected to a source of gaseous medium, the first flow duct extending from an suction opening to the chamber to flow gaseous medium over a culture of prokaryotes housed within the chamber.
